(19) United States
(12) Patent Application Publication
(10) Pub. No.: US 2022/0296821 A1
(43) Pub. Date: Sep. 22, 2022

(54) PEN NEEDLE APPARATUS

(71) Applicant: Embecta Corp., Andover, MA (US)

(72) Inventors: David Poganski, Midland Park, NJ (US); Peter Girgis, East Brunswick, NJ (US)

(73) Assignee: Embecta Corp., Andover, MA (US)

(21) Appl. No.: 17/836,632

(22) Filed: Jun. 9, 2022

Related U.S. Application Data
(63) Continuation of application No. PCT/US2020/063528, filed on Dec. 7, 2020.
(60) Provisional application No. 62/947,140, filed on Dec. 12, 2019.

(51) Int. Cl. A61M 5/34; A61M 5/32; A61M 5/28

(52) U.S. Cl. CPC A61M 5/344; A61M 5/3297; A61M 5/288; A61M 5/3293

(57) ABSTRACT

A pen needle is disclosed for coupling to a delivery device and piercing a septum on the delivery device. The pen needle includes a collar having a side wall, an open proximal end, and a distal end having a coupling member, a needle hub rotatable independently of the collar, where the proximal end of the needle hub has a coupling member coupled to the distal end of the collar. The collar rotates independently from the needle hub so that the collar screws onto the threaded end of the delivery device while the needle hub does not rotate and the needles advance to pierce a septum of the delivery device without rotating the hub and the needles.

### RELATED APPLICATIONS

This application is a continuation patent application of PCT International Patent Application No. PCT/US2020/063528 filed on Dec. 7, 2020, which claims priority to and the benefit of U.S. Provisional Patent Application Ser. No. 62/947,140 filed on Dec. 12, 2019, each of which are hereby incorporated by reference in their entireties.

### FIELD OF THE INVENTION

The present invention relates generally to a pen needle for coupling to a delivery device, such as a pen needle delivery device. The pen needle includes a plurality of needles or cannulas for delivering the substance to a patient and for piercing a septum of the delivery device. The pen needle is configured for coupling to the delivery device without rotating the needles or cannulas relative to the septum when the proximal end of the cannulas or needles pierce the septum of the delivery device.

### BACKGROUND OF THE INVENTION

Insulin and other injectable medications are commonly delivered with drug delivery pens, where a disposable pen needle hub is attached to the pen to facilitate access to the drug container and allow fluid egress from the container through the needle into the patient.

Various pen needle delivery devices are known in the art for dispensing the substance to the patient. The delivery devices often use a disposable needle hub having a cannula or needle extending from a patient end of the hub for inserting into the patient. A non-patient end of the hub is coupled to the pen delivery device for delivering the substance to the patient.

The needle hub assembly is often packaged in a container containing several loose needle hubs. A needle hub is selected from the package and attached to the pen needle delivery device for injecting the patient and then removed to be discarded. The needle hub package includes an outer cover that encloses the needle hub and a removable seal that is peeled from the outer cover to open the cavity so that the needle hub can be removed. The needle hub can have threaded non-patient end that is threaded onto the delivery device. The delivery device with the attached needle hub is then removed from the outer cover. An inner needle shield is attached to the needle hub to cover the cannula until the device is ready for use. The shield is removed to expose the cannula for use to deliver the substance to the patient. After use, the needle hub can be inserted back into the outer cover to enclose the exposed cannula. The pen delivery device is separated from the needle hub leaving the needle hub within the outer cover.

The pen needles often include a needle that extends from the proximal, non-patient end of the needle hub. The needle is positioned to pierce the septum of the delivery pen to access the drug or other medication. The pen needle is threaded onto the end of the delivery pen so that the needle pierces the septum as the needle hub advances onto the delivery pen. The needle typically has a thin diameter which can bend during the insertion into and though the septum, which interferes with the delivery of the drug to the patient.

Existing pen needle assemblies are disclosed in U.S. Patent Application Publication Nos. 2006/0229562 to Marsh et al. and 2007/0149924 to R. Marsh, the entire contents of both of which are hereby incorporated by reference.

Microneedles devices are known having a needle length of less than 5 mm for certain applications. The delivery device can have a single needle or multiple needles arranged in a selected pattern for introducing the medication to a patient.

Although the prior devices have been suitable for the intended use, there is a continuing need in the industry for improved pen needle devices and methods of connecting the pen needle to the delivery device.

### SUMMARY OF THE INVENTION

The present invention relates generally to a pen needle for coupling to a delivery device such as a pen needle delivery device used for injecting a medication to a patient, such as insulin delivery injections. The pen needle includes a plurality of needles or cannulas for delivering the medication or substance to a patient. The pen needle includes a needle hub supporting the needles and a collar that is able to rotate with respect the needle hub. The collar has internal threads for coupling to the delivery device. The collar is able to rotate relative the needle hub so that the plurality of needles engage the septum in a linear direction and resist rotation of the needle hub and resist lateral force against the proximal end of the needles to prevent shearing, bending or damage to the proximal end of the needles.

The pen needle in one embodiment includes a hub with a plurality of needles extending through the hub and having a distal end for injecting and delivering a medication into a patient and a proximal end forming a non-patient end extending from a non-patient proximal end of the hub for piercing a septum of the delivery device to access the medication or other substance to be delivered to the patient. The pen needle is configured for coupling to the delivery device without rotating the needle hub with respect to the delivery device where the needle hub and the needles move in an axial direction so that the needles pierce the septum of the delivery device in an axial direction.

The needles are spaced apart a distance to provide multiple simultaneous injections to deliver the medication to the patient at a controlled depth and rate of delivery. The needles have a suitable exposed distal end extending from the distal end of the needle hub with a selected length and gauge depending on the desired depth of penetration and the volume of the medication to be delivered in a selected length of time. The needles in one embodiment have length of about 1 to 5 mm. In one embodiment, the exposed length of the distal end of the needles is about 2 to 10 mm. In one embodiment, the exposed distal end of the needle has a length of about 2-7 mm. In other embodiments, the needles can have a length of less than about 2 mm. In further embodiments, the needles have an exposed length extending from the distal end of the hub of about 1-2 mm where the needles have a length to deliver a medication subcutaneously or into the epidermis. The needles have a length to provide the exposed proximal end extending from the proximal end of the needle hub to enable the needles to pierce the septum of the delivery device to access the medication of the delivery device. The proximal end of the needles has a length of about 4 to 7 mm.

The gauge of the needles is typically about 32 gauge or smaller. In one embodiment, the needles are 34 gauge. The needles have a gauge that enables the distal end to pierce the skin of the patient a selected depth to deliver the medication and that enables the proximal end to pierce the septum without damaging or bending the needles during the penetration of the septum.

In one embodiment, the pen needle has a threaded collar and a needle hub where the collar can rotate relative to the needle hub supporting a plurality of needles. In one embodiment the needles extend through the hub and project from the distal end and from the proximal end of the hub. The collar is threaded onto a threaded end of the delivery device while the needle hub is configured to remain rotationally fixed relative to the collar and relative to the delivery device. The needle hub moves axially by the rotation of the threaded collar whereby the proximal, non-patient end of the needles move axially to pierce the septum of a delivery device without the needles rotating relative to the septum and the collar.

The needle hub includes a coupling to attach the needle hub to the collar where the collar can rotate independently of the needle hub. In one embodiment, the coupling between the collar and the needle hub is a flange on one of the needle hub or collar that mate with a recess on the other of the needle hub or collar. The collar can have an open end defined by an inwardly extending flange that is received in an annular recess in the needle hub.

In another embodiment, the coupling can be two spaced apart flanges extending outwardly from the needle hub to define an annular recess. The top distal end of the collar is received in the annular recess of the needle hub to allow rotation of the collar relative to the needle hub.

In a further embodiment, the coupling is a flange on the needle hub and at least one tab and typically a plurality of tabs projecting outwardly from the needle hub and spaced from the flange. The space between the flange and the tab captures the top end of the collar so that the collar can rotate relative to the needle hub.

The features of the pen needle are basically obtained by providing the pen needle with a collar having a side wall, an open proximal end, and a distal end, and a needle hub rotatably coupled to the distal end of the collar. The needle hub has a proximal end and a distal end. A plurality of needles are coupled to the needle hub where a distal end projects from the distal end of the needle hub and where a proximal, non-patient end of the needles extends from the proximal distal end of the needle hub. The proximal end of the needles are configured for piercing a septum of a delivery device when the pen needle is connected to the delivery device. The collar is rotatable independently of the needle hub and the needles so that the needle hub can remain rotationally fixed when the collar is rotated and attached to the delivery device. The needle hub is able to move axially with the axial movement of the collar where the proximal end of the needles pierce the septum of the delivery device.

The features are further obtained by providing a pen needle with a collar and a needle hub where the collar has a sidewall, an open proximal end having a coupling member, and a distal end. The needle hub has a proximal end and a distal end with a convex skin-contact surface with a proximal area at an outer edge of the skin-contact surface and a distal area centrally located and spaced axially from the proximal area. The proximal end of the needle hub has a coupling member coupled to the distal end of the collar where the collar is rotatable independently of the needle hub. A plurality of needles are coupled to the needle hub and have a distal end extending from the distal end of the needle hub, and a proximal end extending from the proximal end of the needle hub. The proximal end of the needles are configured for piercing a septum of a delivery device when the pen needle is attached to the delivery device.

The features of the invention are also obtained by providing a method of assembling a pen needle to a pen needle delivery device. The method comprises inserting the pen needle delivery device into a pen needle, where the pen needle includes a threaded collar having a proximal end and distal end, and a needle hub having a proximal end rotatably coupled to the distal end of the collar and a distal end with a plurality of needles extending from the distal end. The proximal end of the needles extend from the proximal end of the needle hub toward the proximal end of the needle hub. The collar rotates relative to the pen needle delivery device without rotating the needle hub so that the proximal end of the needles are able to pierce a septum on the pen needle delivery device

The objects, advantages, and salient features of the invention will become apparent from the following detailed description, which, taken in conjunction with the annexed drawings, discloses exemplary embodiments of the invention.

### BRIEF DESCRIPTION OF THE DRAWINGS

The above benefits and other advantages of the various embodiments of the present invention will be more apparent from the following detailed description of exemplary embodiments of the present invention and from the accompanying figures, in which:

FIG. 1 is an exploded perspective view of a pen needle delivery device showing a pen needle assembly that includes a needle hub supporting a cannula, inner shield, and outer cover;

FIG. 2 is a cross-sectional view of the pen needle of FIG. 1;

FIG. 3 is a perspective view of the pen needle in one embodiment;

FIG. 4 is a perspective view in cross-section of the pen needle of FIG. 3;

FIG. 5 is a cross-sectional side view of the pen needle of FIG. 3 showing the collar and needle hub coupled to the delivery device;

FIG. 6 is a perspective view of the collar of the pen needle of FIG. 3;

FIG. 7 is a perspective view of the needle hub of FIG. 3;

FIG. 8 is an end view of the needle hub of FIG. 3;

FIG. 9 is a cross-sectional view of the needle hub in another embodiment;

FIG. 10 is a perspective view of the pen needle in another embodiment; and

FIG. 11 is cross-sectional view of the pen needle of FIG. 10.

Throughout the drawings, like reference numbers will be understood to refer to like parts, components, and structures.

### DETAILED DESCRIPTION OF THE EXEMPLARY EMBODIMENTS

The present invention is directed to a pen needle for connecting to a delivery device such as a pen needle delivery device. The pen needle supports a plurality of needles or cannulas for delivering a substance to the patient where a proximal, non-patient end of the needles or cannulas are configured for piercing a septum on the delivery device when the pen needle is attached to the delivery device. Pen needle is configured so that a needle hub supporting the needles or cannulas move in a linear direction substantially without rotation of the needle hub relative to the delivery device for piercing the septum on the delivery device when the pen needle is attached to the delivery device.

Reference to embodiments of the present invention are illustrated in the accompanying drawings, wherein like reference numerals refer to like elements throughout. The embodiments described herein exemplify, but do not limit, the present invention by referring to the drawings. The exemplary embodiments are presented in separate descriptions, although the individual features and construction of these embodiments can be combined in any number of ways to meet the needs of the user.

It will be understood by one skilled in the art that this disclosure is not limited in its application to the details of construction and the arrangement of components set forth in the following description or illustrated in the drawings. The embodiments herein are capable of being modified, practiced or carried out in various ways. The term cannula and needle are used interchangeably. For purposes of this disclosure the term needle is used although it is understood that the term includes other devices such as a cannula that is able to pierce a patient for injecting and delivering a medication or other substance to a patient. It will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. Further, terms such as up, down, bottom, and top are relative, and are employed to aid illustration, but are not limiting. The term “substantially” and “about” numerical ranges are understood as including a range that can encompass the specific or give value but not limited to the specific value and can encompass a range of equivalents. The term substantially can encompass a given value or characteristic and a value or characteristic that provides the intended function and/or purpose the value or characteristic. Features of the different embodiments can be combined with features of other embodiments so long as they are not inconsistent with each other.

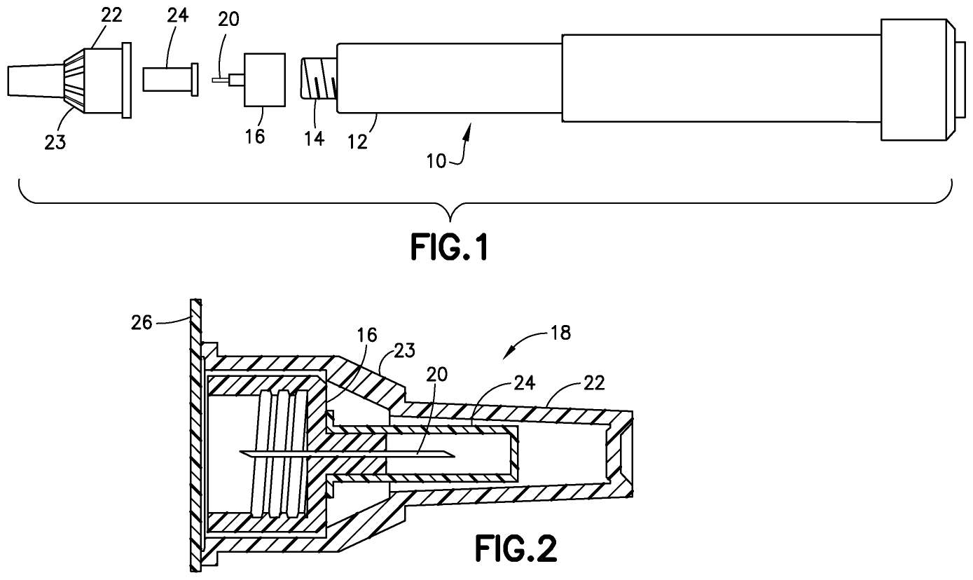

Pen needle delivery device 10, as shown in FIG. 1 typically comprises a dose knob/button, an outer sleeve 12, and a cap. A dose knob/button allows a user to set the dosage of medication to be injected. The outer sleeve 12 is gripped by the user when injecting medication. The cap is used by the user to securely hold the pen needle device 10 in a shirt pocket or other suitable location and provide cover/protection from accidental needle injury.

In standard pen needle devices the dosing and delivery mechanisms are all found within the outer sleeve 12 and is not described in greater detail as they are understood by those knowledgeable of the prior art. A medicament cartridge is typically attached to a standard pen injector housing by known attachment means. The distal movement of a plunger or stopper within the medicament cartridge forces the medication into the reservoir housing. The medicament cartridge is sealed by a septum and punctured by a septum penetrating needle cannula located within a reservoir or housing. Reservoir housing is preferably screwed onto the medicament cartridge although other attachment mechanism can be used. The pen needle delivery device can be a standard pen delivery device known in the industry so that the pen needle delivery device is not shown in detail. The pen needle 18 as shown in FIG. 2 includes a needle hub 16 supporting a cannula 20, an outer cover 22, and an inner shield 24. A protective seal 26 is attached to the open end of the outer cover as shown in FIG. 2 to enclose the needle hub and cannula to maintain a clean and sterile condition. The seal 26 can be a label or other closure member that can be easily peeled from the outer cover to access the needle hub during use.

The pen needle delivery device 10 is connected to needle hub 16 shown in FIG. 1 that has a connecting non-patient end with internal threads that screw onto a threaded end 14 of the delivery device 10. The needle 20 extends from the patient end of the needle hub 16 for delivering the substance to the patient. The outer cover 22 can be provided to cover the needle cannula to protect the patient from accidental needle stick before and after use. The outer cover 22 includes ribs 23 to assist in gripping the outer cover during use. The inner shield 24 is provided over a post extending from the end of the needle hub 16 to enclose the cannula. During use, the needle hub 16 is connected to the pen delivery device and the inner shield is removed. After use, the outer cover is generally placed back on needle hub to cover the needle cannula. The needle hub with the cover is then removed from the pen needle delivery device and discarded.

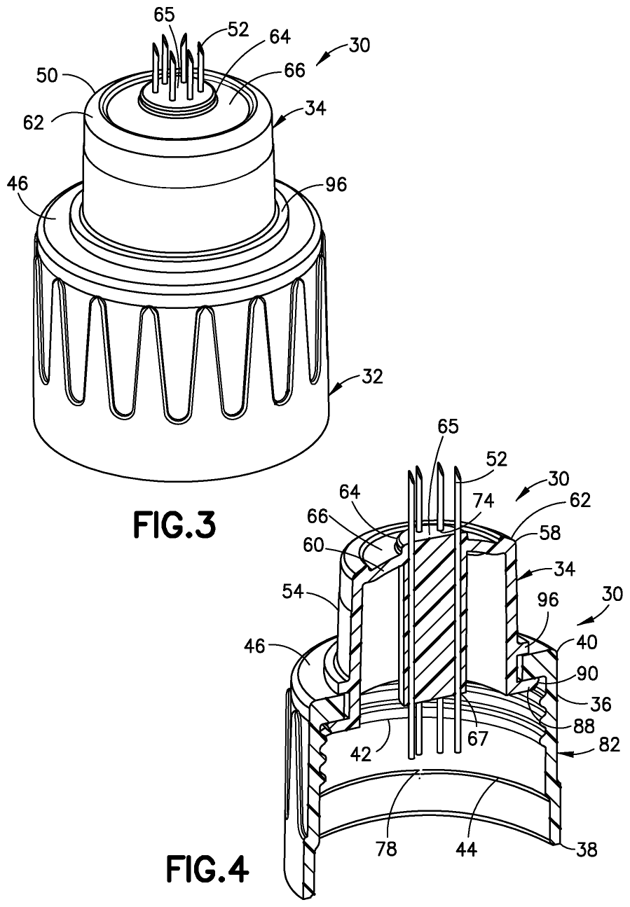

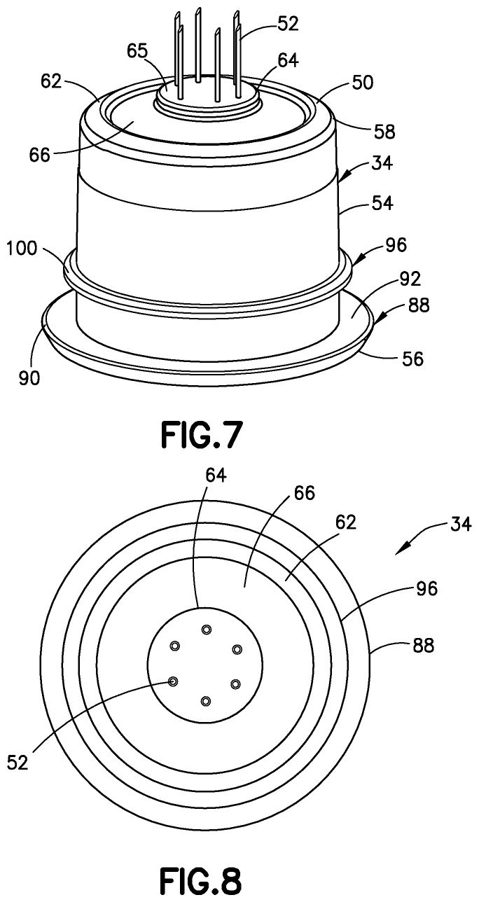

Referring to FIGS. 3-8, a first embodiment of the pen needle is shown. The pen needle 30 in the embodiment shown includes a collar 32 and a needle hub 34. The needle hub 34 is coupled to the collar 32 by a suitable coupling mechanism so that the collar 32 can rotate freely about a longitudinal axis relative to the needle hub 34. The needle hub 34 and collar 32 are typically made of a rigid plastic material. In the embodiment shown, the needle hub 34 and collar 32 are made as separate elements that are assembled to form the pen needle 30.

As shown in FIGS. 4-8, the collar 32 is configured for connecting to the end of the delivery device such as the pen needle delivery device of FIG. 1. In the embodiment shown, the collar 32 has a sidewall 36 forming an open proximal end 38 and a distal end 40. The proximal end is open for coupling with the delivery device as shown in FIG. 4. The sidewall 36 has an inner surface with internal threads 42 for connecting with the delivery device. A chamfered edge 44 as shown in FIG. 4 forms a seal with the delivery device and forms a slightly larger opening for ease of insertion of the delivery device into the open end of the collar 32.

The distal end 40 of the sidewall 36 includes an end wall 46. In the embodiment shown, the end wall 46 extends inwardly from the sidewall 36 substantially perpendicular to the longitudinal axis of the collar 32. The end wall 46 has an inner edge 48 defining an opening 50 in the distal end 40 of the collar 32 as shown in FIG. 6.

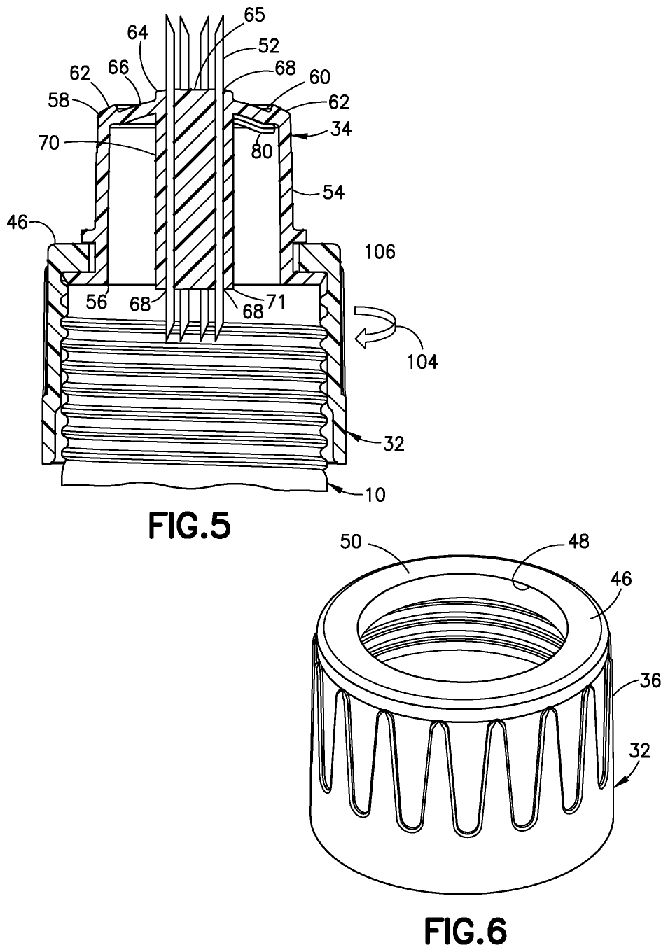

The needle hub 34 has a shape and configuration for supporting a plurality of needles 52 as shown in FIGS. 4 and 5. The needles 52 define a member that is able to pierce the skin of the patient to deliver a medication to a patient. The needles have a lumen and a sharpened distal end for delivering the medication subcutaneously or into the epidermis. The needles are typically made of stainless steel although other materials can be used.

The needles 52 have a length and gauge that can be used in pen needle devices. In one embodiment, the needle can have a distal end with an exposed length of about 1-10 mm although exposed length can be selected according to the intended use and intended depth of penetration into the skin of the patient. In one embodiment, the exposed length of the needle can be about 2-4 mm. In another embodiment, the exposed length of the needle can be about 1-5 mm. In one embodiment, the needle has an exposed distal end length of less than 2 mm. In the embodiment shown, the distal end of the needles has an exposed portion with a substantially uniform and equal length. In other embodiments, the needles can have different lengths to provide different injection depths into the skin for delivery of the medication.

In one embodiment, the needles are 34 gauge to reduce the discomfort of inserting the needles into the patient and reduce the perceived pain. The 34 gauge needles have smaller diameter lumen that can restrict the flow rate of the medication compared to larger diameter needles. The array of needles provides multiple delivery passages from the delivery device to deliver a standard dosage in a relatively short time without increasing the flow rate or pressure from the delivery device. The multiple needles also provide a delivery area that is larger than a single needle to promote improved delivery and absorption by the patient.

The needles 52 in the embodiment shown have a length to extend entirely though the needle hub to have an exposed distal end extending from the distal end of the needle hub with an exposed length for injecting a medication into the patient, and a proximal end extending from the proximal end of the needle hub with a length for piercing the septum. The needles in the embodiment shown in FIG. 4 are a single needle having a length to extend through the needle hub to provide the distal end with a length sufficient to deliver the medication to the patient and a proximal end with a length sufficient to pierce the septum. The needles as shown in the embodiment of FIG. 4 have a substantially uniform diameter and/or gauge along the entire length of the needle.

In other embodiments, the distal end of the needle can have a diameter and/or gauge that is different from the proximal end. In this manner the distal end can have a needle gauge to provide the delivery of the medication to the patient with reduced discomfort to the patient. The proximal end of the needle can have larger diameter to resist bending or shearing when piercing the septum while providing the desired fluid flow of medication from the delivery device to the distal end of the needle.

The plurality of needles 52 are arranged on the distal end of the needle hub 34 to provide a selected needle pattern or array. The needles 52 are spaced apart a distance to enhance the injection of the medication into the patient without interfering without the penetration of an adjacent needle into the patient. In the embodiment shown, the needles 52 are arranged in a cluster, group or array that is substantially symmetrical so that the needles are spaced apart a substantially uniform distance. The dimensions of the pattern are selected to enable sufficient penetration of each of the needles into the patient. In one embodiment, the dimensions of the pattern correspond substantially to the dimensions of the distal face of the needle hub. In the embodiment show, the needles are arranged in an array having a substantially circular configuration having a diameter of about 2-4 mm and a surface area on the distal end of the needle hub of about 2-12 mm². In one embodiment, the needle array covers a surface area on the distal face of the needle hub of about 2-4 mm². In another embodiment, the surface area of the needle array has a diameter of about 3 to 5 mm. As shown, the needles are spaced apart laterally relative to the longitudinal axis of the needle and the needle hub.

In the embodiment shown, the needle hub 34 is formed by a sidewall 54 having an open proximal end 56 and a closed distal end 58. In the embodiment shown, the sidewall 54 has a substantially cylindrical configuration although other shapes and dimensions can be used. The distal end 58 of the needle hub 34 has an end wall 60 closing the distal end of the needle hub and defining a substantially convex skin contact surface for contacting the patient and limiting the depth of penetration of the needle during the delivery of the medication into the patient. In the embodiment shown, the end wall 60 has a distal face with a raised annular outer ring 62 and a raised inner circular shaped projection 64 spaced axially in a distal direction and spaced radially inward from the annular outer ring 62 forming a skin contact surface. A recessed area 66 is formed between the outer ring 62 and the inner projection 64 forming part of the skin contact surface of the needle hub. The contour and position of the inner and outer rings relative to each other and the recessed area 66 define a convex surface having a radius of curvature of about 4-8 mm to control the deformation of the skin when the needle is inserted into the patient and the skin contact surface is pressed against the skin by a common insertion force by the user. As shown in FIG. 4, the outer ring 62, inner projection 64 and recessed area 66 form a conical, convex shaped distal surface.

In one embodiment, the contoured skin contact surface can have a radius of curvature of about 5-8 mm. In another embodiment, the distal face of the needle nub can have a radius of curvature of about 6-8 mm and a width of about 6-8 mm. The ratio of the diameter to the axial length of the distal surface of the needle hub forming the skin contact surface can be about 5:1 to about 8:1. The inner projection 64 can have an axial length of about 0.5 to 1.0 mm and a diameter of 2-3 mm with a surface area of about 3-5 mm². The diameter of the skin contact surface of the distal end of the needle hub can be about 2 to 3 times the diameter of the inner projection.

The outer annular ring 62 has a distal surface forming a proximal area of the skin contact surface with a substantially conical shape corresponding to the radius of curvature of the skin contact surface of the needle hub. The projection 64 is spaced axially outward and radially inward from the annular outer ring 62 and has a dimension to support the needles in an array with a selected pattern to obtain the desired delivery and depth of penetration of the medication. The projection 64 has an outer surface forming a distal surface area 65 of the skin contact surface for receiving the needle array. The distal surface area 65 of the projection can have convex shape or a substantially flat surface extending in a plane perpendicular to the longitudinal axis of the needle hub.

The distal surface area 65 of the projection 64 is configured to support a selected number of needles in a selected array or pattern. In the embodiment shown, six needles are provided although the number of needles can vary depending on the medication, the desired volume of delivery, the desired depth of penetration, and the rate of delivery. The number of needles can range from about 4-12 and typically about 5-12. In one embodiment, the number needles in the array can be at least 5 and typically 6-8 needles.

The needles can be arranged in a suitable array or pattern to achieve the desired penetration into the patient and the desired delivery of the medication. As shown the drawings, the needles can be arranged in a substantially circular array or pattern around the distal face 65 of the projection 64. The needles are generally spaced apart a uniform distance from an adjacent needle and spaced inward from the peripheral edge of the post.

The distal face 65 of the projection 64 is generally circular and has a diameter of about 1-4 mm and generally a diameter of about 1.5-3.0 mm. The surface area of the distal face of the projection 64 can be about 3 to about 7 mm². The area defined by the needle array can be about 3 to 7 mm² and generally about 3-5 mm².

The end wall 60 has a plurality of spaced apart axial passages 68 with a dimension for receiving a needle. The needles can be coupled to the needle hub by an adhesive, friction fit or other suitable attachment mechanism. The proximal end of the needles has a length greater than the longitudinal dimension of the side wall 54 of the needle hub 34 so that the proximal end of the needles extends into the collar as shown FIG. 4.

Referring to FIG. 4 a needle post 70 extends from an inner surface 68 of the end wall 60 toward the proximal end of the needle hub. The post 70 as shown has a substantially cylindrical shape with a distal end formed with an end wall 60 and a proximal end 71. The axial passages 68 for receiving the needles 52 extend between the distal face 65 of the projection 64 and the proximal end 71 of the post 70. Radially extending ribs 80 are formed on the inner surface of the end wall 60 as shown in FIG. 4 to assist in stabilizing the post 70 and strengthening the end wall 60. The post 70 in the embodiment shown is integrally formed with the end wall of the needle hub as in a one-piece molded unit. In other embodiments, the projection can be a separate member that is attached to the needle hub by a suitable attachment mechanism. The needle 52 can be attached by a suitable adhesive in the axial passages 68 of the needle hub.

The proximal end 56 of the needle hub 34 is formed with a coupling for connecting the needle hub 34 to the collar 32 while allowing rotation between the needle hub and the collar. The coupling can be a flange on one of the collar and the needle hub and a complementing recess on the other of the collar and the needle hub. In the embodiment shown, the coupling is formed by an annular flange 88 extending radially outward from the proximal end of the sidewall 54 of the needle hub 34. The flange 88 has a peripheral edge 90 complementing the inner dimension of the collar 32. As shown in FIG. 5, the sidewall 54 of the needle hub 34 has a dimension complementing the opening 50 in the collar whereby the needle hub 34 can rotate within the opening in the collar. The flange 88 has a top face 92 with a surface complementing the bottom surface of the end wall 46 of the collar 32. In the embodiment shown, the top face 92 is a substantially flat surface extending radially outward from the longitudinal axis of the needle hub 34. The bottom face of the flange 88 also has a substantially flat radially extending surface for mating with the end of the delivery device 10.

The coupling mechanism in the embodiment shown also includes a second flange 96 extending radially outward from the sidewall 54 of the needle hub 34. The second flange 96 is spaced from the flange 88 a distance to form an annular recess corresponding substantially to the thickness of the end wall 46. The flange 96 has a radial dimension less than the radial dimension of the flange 88. The flange 96 has a top face with a chamfered edge 100. The chamfered edge 100 is able to slide over the inner edge of the opening 50 in the end wall 46 by a snap connection to couple the needle hub 34 to the collar 32. As shown in FIG. 4, the needle hub 34 is coupled to the collar 32 by the end wall 46 captured in the recess between the flange 88 of the flange 96. The collar 32 and the hub 34 are connected together so that the needle hub can rotate freely in the open end of the collar 32.

The pen needle 30 is connected to the threaded end of the delivery device 10 as shown in FIG. 5. The collar 32 is rotated as indicated by arrow 104 shown in FIG. 5 to advance the pen needle onto the delivery device 10. The collar 32 rotates to screw the collar onto the threaded end of the delivery device 10. The needle hub 34 is able to remain rotationally fixed relative to the collar 34 so that the needle hub 34 moves toward the septum in a linear direction substantially without rotational movement of the needle hub 34. The needles 52 are able to pierce the septum of the delivery device 10 by moving in a substantially linear direction without twisting or rotation of the post or projection relative to the septum of the delivery device. Screwing the collar onto the delivery device enables the needle 52 to pierce the septum to access the contents of the delivery device and provide the fluid communication between the delivery device and the needle 52. In one embodiment, the end of each needle 52 is spaced inwardly from the proximal end of the collar 32 so that the threads of the collar engage the threads of the delivery device before the needles engage the septum. Rotating the collar 32 relative to the delivery device 10 moves the needle hub and needles into engagement with the septum.

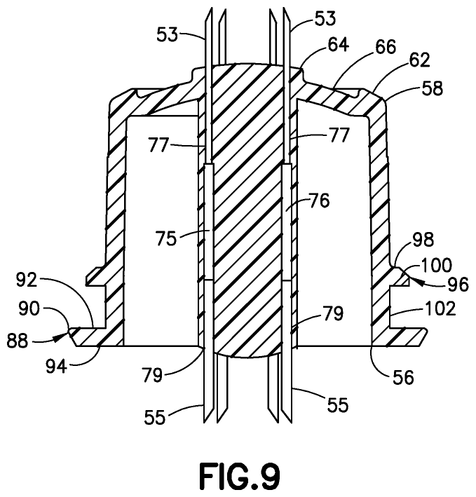

In another embodiment shown in FIG. 9, the needle hub 34 can have a plurality of axial passages 75 as in the previous embodiment where the needles are defined by a distal needle 53 coupled to the distal end of the needle hub 34 for delivering the medication to a patient and a proximal needle coupled to the proximal end 55 of the needle hub for piercing the septum of the delivery device. The separate proximal needle and distal needle can be the same or different. The axial passages 75 in the embodiment shown have a distal section 77 having a dimension for receiving the distal needle 53 and a proximal section 79 having a dimension for receiving the proximal needle 55. In the embodiment shown, the proximal needle can have a larger diameter to enable the proximal needle to pierce the septum with reduced risk of bending or shearing when piercing the septum while providing sufficient flow of the medication through the proximal needles to the axially passage of the needle hub. The axial passage 75 carries the medication from the proximal needle 55 to the proximal end of the distal needle 53. The distal needle 53 in the embodiment shown has a smaller diameter, such as a 34 gauge, to deliver the medication with reduced pain or discomfort to the patient while the proximal needle 55 can be 32 gauge.

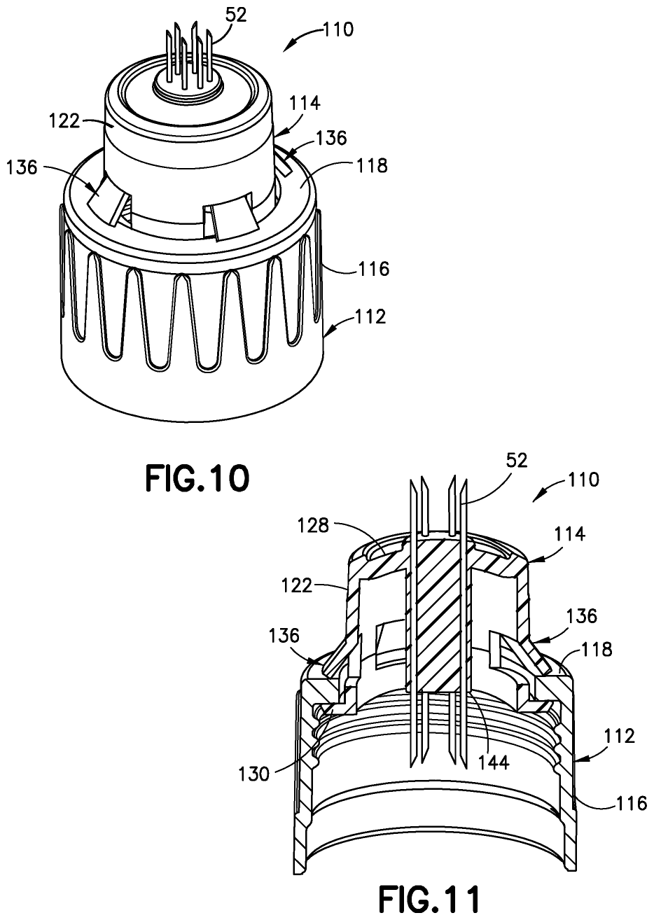

In another embodiment shown in FIGS. 10 and 11, a pen needle 110 includes a collar 112 and a needle hub 114. Collar 112 is substantially the same as in the embodiment of FIGS. 3-8 where the collar 112 includes a sidewall 116, and an end wall 118 forming an inwardly extending flange with an inner edge forming an opening.

Needle hub 114 is similar to the needle hub in the previous embodiment having a sidewall 122 with a substantially cylindrical configuration with a proximal end and a distal end. An end wall 128 is formed at the distal end for supporting the needle 52 as in the previous embodiment. The end wall 128 in the embodiment shown has an inner projection and an outer ring forming the skin contact surface in a manner similar to the previous embodiment.

The proximal end of the sidewall 122 includes an outwardly extending flange 130 with a dimension complementing the inner dimension of the collar 112. The flange 130 has a bottom face for mating with the end of the delivery device and a top face for contacting the bottom face of the inwardly extending flange 118 of the collar 112. The needle hub 114 includes a post 144 extending axially from the end wall 128 for supporting the needles 52 as in the previous embodiment. The post 144 is substantially the same as in the previous embodiment.

The coupling between the collar 112 and needle hub 114 in the embodiment shown includes at least one and typically a plurality of tabs 136. The tabs 136 are formed by cutouts in the sidewall 122. As shown in FIG. 10, the tabs 136 are integrally formed with the sidewall 122 and project outwardly at an incline from the sidewall towards the proximal end of the needle hub and form an annular recess between the end of the tabs 136 and the flange 130. The tabs 136 have a first end connected to the sidewall and a free end for contacting the flange 118 of the collar 112. The tabs 136 are sufficiently flexible so that the needle hub can be connected to the collar 112 by sliding the needle hub through the opening in the collar whereby the tabs 136 are able to slide past the end wall or flange 118 of the collar 112 and snap outwardly to the position shown in FIG. 10. In the embodiment shown, the needle hub includes four tabs 136 although the number of tabs can be more or less than four as needed to retain the needle hub on the collar without inhibiting rotation of the needle hub relative to the collar.

The foregoing embodiments and advantages are exemplary and are not to be construed as limiting the scope of the present invention. The description of an exemplary embodiment of the present invention is intended to be illustrative, and not to limit the scope of the present invention. Various modifications, alternatives, and variations will be apparent to those of ordinary skill in the art, and are intended to fall within the scope of the invention. It is particularly noted that the features of different embodiments and claims may be combined with each other as long as they do not contradict each other. Accordingly, all such modifications are intended to be included within the scope of this invention as defined in the appended claims and their equivalents.

1. A pen needle comprising:
a collar having a side wall, an open proximal end, and a distal end;
a needle hub rotatably coupled to said distal end of said collar, said needle hub having a proximal end and a distal end, and a plurality of needles coupled to said needle hub and having a distal end extending from said distal end of said needle hub, and a proximal end extending from said proximal end of the needle hub, said proximal end of said needles configured for piercing a septum of a delivery device, said distal end of said needles configured for administering a medication to a patient, and where said collar is rotatable independently of said needle hub.

2. The pen needle of claim 1, wherein said needle hub has an end wall at said distal end and post extending from said end wall toward said proximal end, a plurality of axial spaced apart passages extending between said distal end of said needle hub and proximal end of said post, each said axial passage receiving a respective needle.

3. The pen needle of claim 2, wherein said plurality of needles are arranged in a configuration having a diameter of about 3 to 5 mm.

4. The pen needle of claim 3, wherein said needle hub comprises at least five needles.

5. The pen needle of claim 3, wherein said needle hub comprises 5 to 12 needles spaced apart in a substantially uniform pattern.

6. The pen needle of claim 1 wherein said needle hub has a plurality of spaced apart axial passages extending between said distal end and said proximal end, each said needle comprising a distal needle received in a distal end of a respective axial passage and coupled to said needle hub and a proximal needle received in a proximal end of a respective axial passage a proximal end of said needle hub, and where said distal needle is in fluid communication with said proximal needle.

7. The pen needle of claim 6, wherein said distal needle has a first gauge and said proximal needle has a second gauge with an inner diameter larger than an inner diameter of said first gauge.

8. The pen needle of claim 1, where said wall of said collar has an inner surface with threads configured for coupling to the delivery device, said collar has an opening in said distal end, and where said needle hub is rotatable within said opening in said distal end of said collar.

9. The pen needle of claim 1, wherein said distal end of said collar has an end wall with an opening; and where said proximal end of said needle hub has an annular recess receiving said end wall of said collar.

10. The pen needle of claim 7, wherein said needle hub has a first flange at said proximal end, and a second flange spaced from said first flange to define said annular recess of said needle hub between said first flange and second flange.

11. The pen needle of claim 1, wherein said proximal end of said needle hub is configured for mating with a distal end of said delivery device and where said collar has internal threads configured for coupling to a delivery device, where said collar rotates with respect to said delivery device and said needle hub is rotatably fixed with respect to said delivery device.

12. The pen needle of claim 1, wherein said needle hub has a side wall with a proximal end and a distal end, and an end wall at said distal end of said needle hub, where said distal end of said needles extend from an outer surface of said end wall, and said proximal end of said needles extend toward said proximal end of said needle hub.

13. The pen needle of claim 1, wherein said needle hub has a side wall with an outwardly extending flange at said proximal end of said needle hub, and a plurality of outwardly extending tabs spaced from said flange, and where said distal end of said collar is captured between said flange and said plurality of tabs to couple said needle hub to said collar, where said collar is rotatable relative to said needle hub.

14. The pen needle of claim 1, wherein said distal end of said needle hub has a convex skin contact surface with an outer ring forming a proximal area at an outer edge of said skin contact surface and a centrally located projection forming a distal area centrally located on said skin contact surface and spaced axially in a distal direction and spaced radially inward from said outer ring, and where said plurality of needles are oriented in said distal area of said projection.

15. A pen needle comprising:
a collar having a side wall, an open proximal end having a coupling configured for coupling to a delivery device, and a distal end with a central opening;
a needle hub having a proximal end and a distal end, said proximal end of said needle hub having a coupling member coupled to said distal end of said collar where said needle hub is rotatable independently of said collar about a longitudinal axis of said pen needle, said distal end of said needle hub having a convex skin contact surface with an outer ring forming a proximal area at an outer edge of said skin contact surface and a centrally located projection forming a distal area centrally located on said skin contact surface and spaced axially outward and radially inward from said outer ring;
a plurality of spaced apart needles having a distal end extending from said distal area of said needle hub, and a proximal end extending from said proximal end of said needle hub, said proximal end of said needles configured for piercing a septum of the delivery device and having an opening providing fluid communication between the delivery device and said distal end of said needles.

16. The pen needle of claim 15, wherein the needle hub has a side wall and an end wall at said distal end of said needle hub, and a post extending proximally from said end wall where said needles extend proximally from said post.

17. The pen needle of claim 16, wherein said side wall of said needle hub has an outwardly extending first flange at said proximal end, and a coupling member extending outwardly from said side wall of said needle hub and spaced from said first flange forming a recess receiving a flange on said collar extending inwardly toward said central opening.

18. The pen needle of claim 17, wherein said coupling member is a second flange.

19. The pen needle of claim 17, wherein said coupling member is at least one tab projecting outwardly from said side wall and spaced from said first flange.

20. The pen needle of claim 19, wherein said at least one tab is flexible.

21. The pen needle of claim 17, wherein said needles have a uniform diameter extending between said distal end and proximal end.

22. The pen needle of claim 16, wherein said hub has a plurality of axial passages extending between said distal end and proximal end of said post, and where said axial passage at said distal end of said needle hub receives a distal needle having a first diameter, and said axial passage at said proximal end receives a proximal needle having a second diameter larger than said first diameter.
